(19) United States
(12) Patent Application Publication
(10) Pub. No.: US 2025/0010044 A1
(43) Pub. Date: Jan. 9, 2025

(54) ADJUSTABLE SHUNTING SYSTEMS AND ASSOCIATED SYSTEMS, DEVICES, AND METHODS

(71) Applicant: Shifamed Holdings, LLC, Campbell, CA (US)

(72) Inventors: Eric Schultz, Los Altos, CA (US); Robert Chang, Belmont, CA (US); Katherine Sapozhnikov, Campbell, CA (US); Tom Saul, Portland, OR (US); David Batten, San Francisco, CA (US); Tessa Bronez, Campbell, CA (US)

(21) Appl. No.: 18/709,924

(22) PCT Filed: Dec. 6, 2022
(86) PCT No.: PCT/US2022/052002

§ 371 Date: May 14, 2024

Related U.S. Application Data
(60) Provisional application No. 63/286,283, filed on Dec. 6, 2021.
(60) Provisional application No. 63/332,997, filed on Apr. 20, 2022.

(51) Int. Cl. A61M 27/00

(52) U.S. Cl. CPC A61M 27/002; A61M 2205/0266; A61M 2205/3334

(57) ABSTRACT

The present technology is generally directed to adjustable shunting systems, including adjustable shunting systems having at least two discrete fluid flow paths having different fluid resistances. In at least some embodiments, the shunting systems include an actuator that controls which of the two discrete fluid flow paths is “open” to fluid flow. For example, the actuator can be configured to selectively control the flow of fluid through the system by selectively alternating between (i) opening a first flow path while closing a second flow path, and (ii) opening the second flow path while closing the first flow path.

### CROSS-REFERENCE TO RELATED APPLICATION(S)

The present application claims priority to U.S. Provisional Patent Application No. 63/286,283, filed Dec. 6, 2021, and U.S. Provisional Patent Application No. 63/332,997, filed Apr. 20, 2022, the disclosures of which are incorporated by reference herein in their entireties.

### TECHNICAL FIELD

The present technology generally relates to implantable medical devices and, in particular, to adjustable shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen(s)). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided to meet the patient's individual and variable needs and/or account for changes in flow-related characteristics, including the flow rate between the two fluidly connected bodies.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is a partially schematic top view of an adjustable shunting system configured in accordance with embodiments of the present technology.

FIGS. 1B and 1C are respective partially schematic top views of a flow control assembly of the adjustable shunting system of FIG. 1A.

FIG. 1D is an exploded perspective view of the flow control assembly of FIG. 1B.

FIGS. 2A and 2B are circuit diagrams that schematically illustrate flow paths through the adjustable shunting system of FIG. 1A.

### DETAILED DESCRIPTION

The present technology is generally directed to adjustable shunting systems, including adjustable shunting systems having at least two discrete fluid flow paths. In at least some embodiments, the shunting systems include an actuator for selectively controlling which of the two discrete fluid flow paths is “open” to fluid flow. For example, the actuator can be configured to control the flow of fluid through the system by selectively alternating between (i) opening a first flow path while closing a second flow path, and (ii) opening the second flow path while closing the first flow path.

The adjustable systems described herein can include two fluid channels having different fluid resistances, and the actuator can be configured to control the flow of fluid through each of the fluid channels, e.g., by selectively interfering with respective channel inlets of the fluid channels. For example, the actuator can be transitioned between (i) a first configuration in which the actuator interferes with and/or at least partially blocks the flow of fluid through a first fluid channel and (ii) a second configuration in which the actuator interferes with and/or at least partially blocks the flow of fluid through a second fluid channel. In some embodiments, the actuator permits (e.g., does not block) fluid flow through the second channel when in the first configuration. Likewise, in some embodiments the actuator permits (e.g., does not block) fluid flow through the first channel when in the second configuration. In such embodiments, the adjustable shunting systems are expected to have at least one open flow path and at least one partially blocked flow path at any given time.

As described in greater detail below, it is expected that in at least some embodiments the present technology may exhibit one or more advantageous characteristics that improve operation of adjustable shunting systems. For example, using a single actuator to control the flow of fluid through multiple flow channels is expected to advantageously reduce the overall size of the system, as compared with systems that have a separate actuator for each flow channel. This may be beneficial in embodiments in which the system is designed to be implanted in certain locations, such as within a patient's eye.

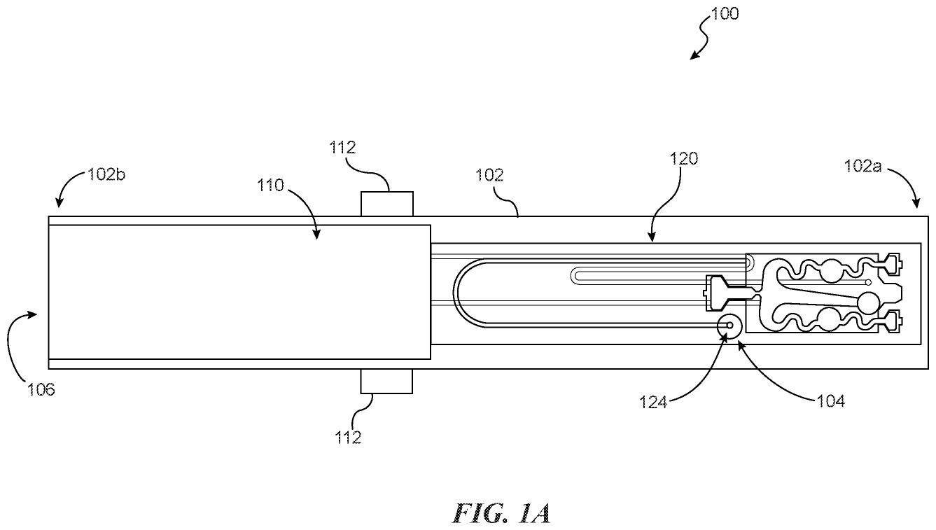

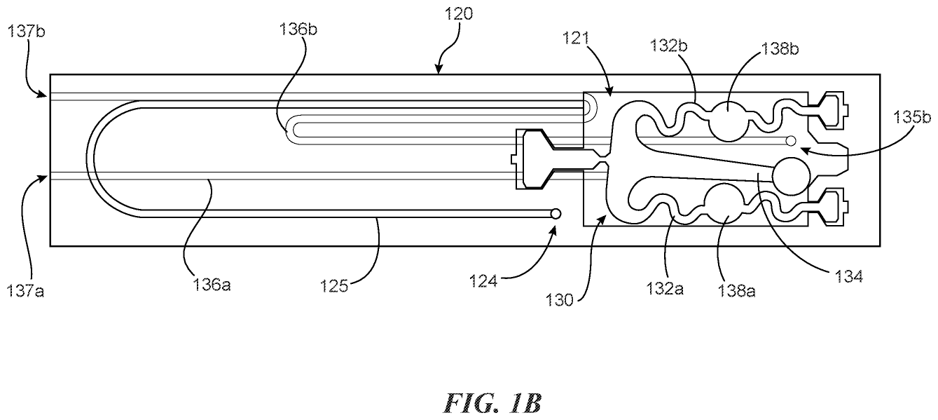

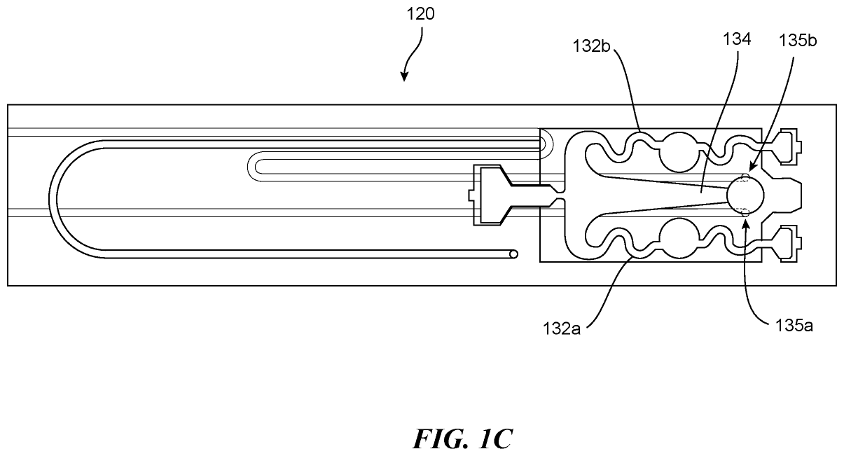

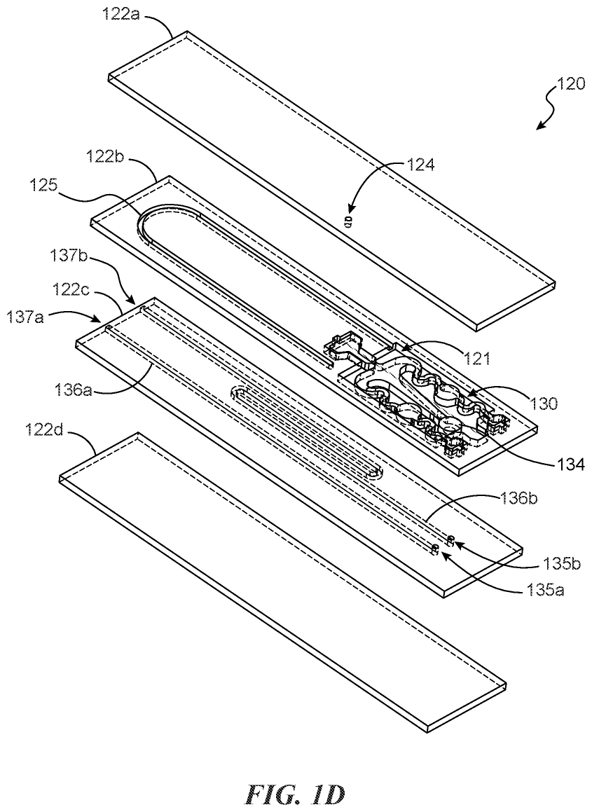

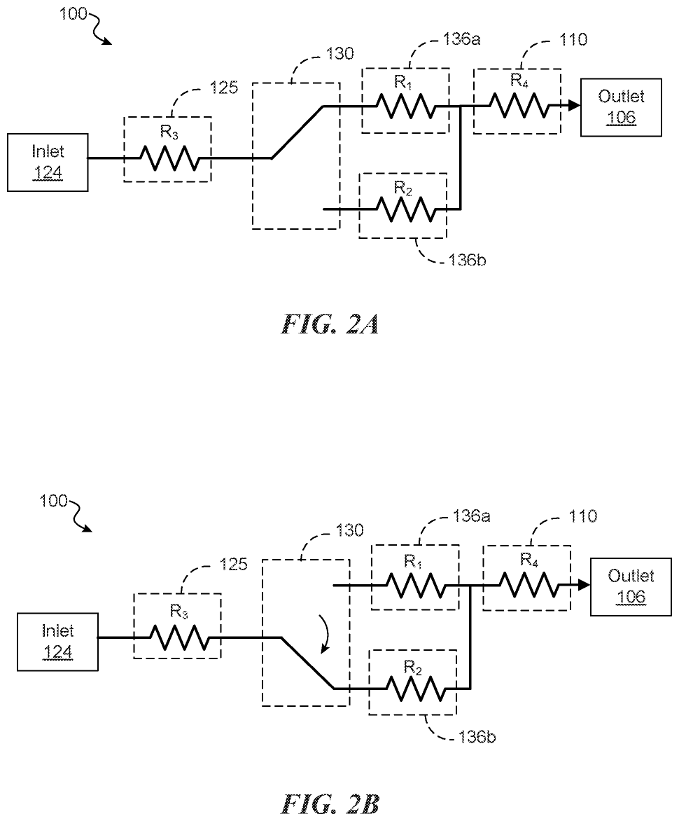

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-2B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics described herein may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIG. 1A is a partially schematic top view of a shunting system 100 (“the system 100”) configured in accordance with embodiments of the present technology. As described in greater detail below, the system 100 is configured to provide an adjustable therapy for draining fluid from a first body region to a second, different body region of a patient, such as to drain aqueous from an anterior chamber of the patient's eye.

The system 100 includes a generally elongated housing 102 and a flow control assembly 120. The elongated housing 102 (which can also be referred to as a casing, membrane, shunting element, or the like) extends between a first end portion 102a and a second end portion 102b. The flow control assembly 120 (which can also be referred to as a flow control plate, a flow control cartridge, a plate structure, a plate assembly, or the like) is positioned within the elongated housing 102 and is configured to selectively control the flow of fluid through the system 100, as described in detail below with reference to FIG. 1B. In the illustrated embodiment, the elongated housing 102 includes an opening 104 that aligns with a fluid aperture or inlet 124 in the flow control assembly 120, as described in further detail below with respect to FIG. 1B. In some embodiments, an outer surface of the flow control assembly 120 forms a substantial fluid seal with an inner surface of the elongated housing 102, such that fluid flowing into the system 100 via the opening 104 generally must pass through the flow control assembly 120. The elongated housing 102 further includes a main fluid conduit 110 fluidly coupling the flow control assembly 120 to one or more fluid outlets 106 positioned proximate the second end portion 102b of the elongated housing 102. In some embodiments, the elongated housing 102 is composed of a slightly elastic or flexible biocompatible material (e.g., silicone, etc.). The elongated housing 102 can also optionally have one or more wings or appendages 112 for securing the elongated housing 102 in a desired position within a patient.

FIG. 1B is a partially schematic top view of the flow control assembly 120 of the adjustable shunting system of FIG. TA. As noted above, the flow control assembly 120 can at least partially define one or more fluid paths through the system 100 (FIG. TA). In the illustrated embodiment, for example, the fluid inlet 124 of the flow control assembly 120 aligns with the opening 104 (FIG. TA) in the elongated housing 102 (FIG. TA). The fluid inlet 124 is in fluid communication with (e.g., provides access to) a fluid inlet conduit 125 that fluidly couples the fluid inlet 124 to a chamber or cavity 121 positioned within an interior of the flow control assembly 120. Accordingly, the fluid inlet 124 can permit fluid to enter the fluid inlet conduit 125 and flow into the chamber 121 of the flow control assembly 120, and thus an interior of the elongated housing 102, from an environment external to the system 100. Although the fluid inlet conduit 125 is illustrated as having a “U” shape in FIGS. 1A-1C, in other embodiments the fluid inlet conduit 125 can have a linear, rectilinear, curved, curvilinear, or any other suitable shape. In further embodiments, the fluid inlet conduit 125 can be omitted and the fluid inlet 124 can be at least partially aligned with the chamber 121 such that fluid can enter the chamber 121 from an environment external to the system 100 directly via the fluid inlet 124.

Fluid can flow out of the chamber 121 via one or more channels 136 extending between the chamber 121 and the main fluid conduit 110 (FIG. TA). Each of the channels 136 can be fluidly isolated such that each of the channels 136 can define a discrete flow path through the flow control assembly 120. For example, in the illustrated embodiment, the flow control assembly 120 includes a first channel 136a and a second channel 136b, each of which has a respective channel inlet 135 at the chamber 121 (e.g., a first channel inlet 135a (FIGS. 1C and 1D) for the first channel 136a and a second channel inlet 135b for the second channel 136b). Additionally, each of the channels 136a-b can include a respective channel outlet 137 (e.g., a first channel outlet 137a for the first channel 136a and a second channel outlet 137b for the second channel 136b) fluidly coupled to the main fluid conduit 110 (FIG. TA). Fluid that enters the flow control assembly 120 via the fluid inlet 124 flows into the chamber 121 and can drain to the main fluid conduit 110 (FIG. TA) via at least one of the channels 136a-b. Each of the channels 136a-b, and/or respective portions thereof, can also have different geometric configurations and/or dimensions (e.g., lengths, widths, diameters, cross-sectional areas, etc.) relative to one another such that they have different fluid resistances, and thus provide different flow rates for a given pressure. In the illustrated embodiment, for example, the first channel 136a has a first length corresponding to a first fluid resistance and the second channel 136b has a second length greater than the first length and corresponding to a second fluid resistance greater than the first fluid resistance. In other embodiments, the first and second channels 136a-b can have a same length, but the first channel 136a can have a smaller cross-sectional area than the second channel 136b. In further embodiments, the channels 136a-b can have any other suitable configuration relative to each other.

The relative level of therapy provided by each fluid path (e.g., the channels 136a-b) can be different so that a user may adjust the level of therapy provided by the system 100 by selectively opening and/or closing various fluid paths (e.g., by selectively interfering with or permitting flow through individual channel inlets 135a-b), as described below. For example, under a given pressure, when fluid primarily drains through the first channel 136a, the system 100 can provide a first drainage rate, and when fluid primarily drains through the second channel 136b, the system 100 can provide a second drainage rate less than the first drainage rate.

The flow control assembly 120 can be configured to selectively control the flow of fluid through at least a portion of the system 100. In particular, the flow control assembly 120 includes an actuator 130 positioned in the chamber 121 and configured to control the flow of fluid through the first channel inlet 135a of the first channel 136a and the second channel inlet 135b of the second channel 136b. Accordingly, actuators in accordance with various embodiments of the present technology can be configured to control the flow of fluid through a plurality of channel inlets and the associated channels.

The actuator 130 can include a projection or gating element 134. In some embodiments, the actuator 130 and/or the gating element 134 can be positioned between the inlet 124 and the channel inlets 135a-b. In the illustrated embodiment, and as shown in FIGS. 1B and 1C, the gating element 134 is positioned above the channel inlets 135a-b and the associated channels 136a-b, such that the actuator 130 is configured to control the flow of fluid through one or more channels 136a-b positioned beneath the gating element 134. Accordingly, the illustrated gating element 134 is configured to selectively control the flow of fluid that has entered the chamber 121 via the inlet 124, for example, to allow and/or prevent fluid flow from the chamber 121 to the main fluid conduit 110 via one or more of the channels 136a-b. In other embodiments, however, the gating element 134 can be positioned below the channel inlets 135a-b, and/or have any other suitable position relative to the channel inlets 135a-b.

The gating element 134 can be configured to moveably interface with the first channel inlet 135a and the second channel inlet 135b. For example, the actuator 130 can be configured to move between at least (i) a first position or configuration (not shown) in which the gating element 134 permits fluid to flow through the first channel inlet 135a (e.g., by not interfering with the first channel inlet 135a) and substantially prevents fluid from flowing through the second channel inlet 135b (e.g., by blocking the second channel inlet 135b), and (ii) a second position or configuration (shown in FIG. 1B) in which the gating element 134 substantially prevents fluid from flowing through the first channel inlet 135a (e.g., by blocking the first channel inlet 135a) and permits fluid to flow through the second channel inlet 135b (e.g., by not interfering with the second channel inlet 135b). In some embodiments, the gating element 134 can optionally be configured to move to one or more intermediate positions between the first and the second position (e.g., a third position, a fourth position, etc.) in which the first channel inlet 135a and the second channel inlet 135b can each be unblocked, partially blocked, or fully unblocked. In at least some embodiments, for example, the gating element 134 can be configured to move to a third position different than the first and second positions. In the embodiment illustrated in FIG. 1C, for example, the gating element 134 is located at least partially between the first channel inlet 135a and the second channel inlet 135b. When the gating element 134 is in the third position, the first channel inlet 135a and the second channel inlet 135b can each individually be either unblocked, partially blocked (e.g., such as shown in FIG. 1C), or fully unblocked. Additionally, or alternatively, the gating element 134 can optionally be configured to move to one or more additional positions outside of the first position and the second position. In at least some embodiments, for example, the gating element 134 can be configured to move to a third position in which the gating element 134 is positioned on a same side (e.g., left, right, etc.) of the first and second channel inlets 135a-b and/or in which neither of the channel inlets 135a-b are blocked. In embodiments where the system 100 includes more than two channels, individual ones of the one or more intermediate positions can correspond to all or a subset of the more than two channels being unblocked, partially blocked, and/or fully blocked.

In some embodiments, the gating element 134 can include a spherical component or portion, such as a bead or a ball-shaped element, configured to be at least partially aligned with one or both of the first and second channel inlets 135a-b. The spherical component or portion can be configured to sealingly engage or plug the first and/or second channel inlets 135a-b, for example, when aligned with the respective first and/or second channel inlets 135a-b. In at least some embodiments, for example, the gating element 134 can include a spherical portion having a curved or arcuate lower surface shaped to correspond to and/or configured to be received at least partially within one or both of the first and/or second channel inlets 135a-b, such that the curved or arcuate lower surface sealingly engages one or both of the first and/or second channel inlets 135a-b. Additionally, or alternatively, the gating element 134 can include silicone and/or one or more other aspects that are generally similar or identical to at least one of the gating elements described in International Patent Application No. PCT/US21/49140, the disclosure of which is incorporated by reference herein in its entirety and for all purposes. In these and other embodiments, the gating element 134 can include any other suitable material(s) and/or combination(s) thereof. In some aspects, gating elements with spherical portions and/or that include silicone can have improved sealing engagement with the channel inlets 135a-b, for example, to partially or fully prevent fluid from leaking into the channels 136a-b when the gating element 134 engages the corresponding channel inlets 135a-b.

The actuator 130 can further include a first actuation element 132a and a second actuation element 132b that drive movement of the gating element 134 between the first position and the second position. The first actuation element 132a and the second actuation element 132b can be composed at least partially of a shape memory material or alloy (e.g., nitinol). Accordingly, the first actuation element 132a and the second actuation element 132b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 132a and the second actuation element 132b may have reduced (e.g., relatively less stiff) mechanical properties that cause the actuation elements to be more easily deformable (e.g., compressible, expandable, etc.) relative to when the actuation elements are in the second material state. In the second material state, the first actuation element 132a and the second actuation element 132b may have increased (e.g., relatively stiffer) mechanical properties relative to the first material state, causing an increased preference toward a specific preferred geometry (e.g., original geometry, manufactured or fabricated geometry, heat set geometry, etc.). The first actuation element 132a and the second actuation element 132b can be selectively and independently transitioned between the first material state and the second material state by applying energy (e.g., laser energy, electrical energy, etc.) to the first actuation element 132a or the second actuation element 132b to heat it above a transition temperature (e.g., above an austenite finish (Af) temperature, which is generally greater than body temperature). If the first actuation element 132a (or the second actuation element 132b) is deformed relative to its preferred geometry when heated above the transition temperature, the first actuation element 132a (or the second actuation element 132b) will move to and/or toward its preferred geometry. In some embodiments, the first actuation element 132a and the second actuation element 132b are operably coupled such that, when the actuated actuation element (e.g., the first actuation element 132a) transitions toward its preferred geometry, the non-actuated actuation element (e.g., the second actuation element 132b) is further deformed relative to its preferred geometry.

The first and second actuation elements 132a and 132b are configured to move in concert with one another and the gating element 134. In some embodiments the gating element 134 is configured to move toward the actuation element being currently actuated. In other embodiments, however, the gating element 134 is configured to move away from the actuation element being currently actuated. The first actuation element 132a and the second actuation element 132b generally act in opposition. For example, the first actuation element 132a can be actuated to move the gating element 134 to and/or toward the first position, and the second actuation element 132b can be actuated to move the gating element 134 to and/or toward the second position. In other embodiments, the orientation can be reversed, such that the first actuation element 132a can be actuated to move the gating element 134 to and/or toward the second position, and the second actuation element 132b can be actuated to move the gating element to and/or toward the first position. Additionally, as described above, the first actuation element 132a and the second actuation element 132b can be coupled such that as one moves toward its preferred geometry upon material phase transition, the other is deformed relative to its preferred geometry. This enables the actuation elements 132a-b to be repeatedly actuated and the gating element 134 to be repeatedly cycled between the first position and the second position.

As best seen in FIG. 1B, in some embodiments each actuation element 132a-b can include one or more targets 138 (shown as a first target 138a on the first actuation element 132a and a second target 138b on the second actuation element 132b). The target(s) 138a-b can be thermally coupled to the corresponding actuation element(s) 132a-b such that energy (e.g., laser energy) received at the target(s) 138a-b can dissipate through the corresponding actuation element(s) 132a-b as heat. The target(s) 138a-b can therefore be selectively targeted with non-invasive energy to actuate the actuation elements 132a-b. For example, to actuate the first actuation element 132a, heat/energy can be applied to the first target 138a, such as from an energy source positioned external to the patient's eye (e.g., a laser). The heat applied to the first target 138a spreads through at least a portion of the first actuation element 132a, which can heat the first actuation element 132a above its transition temperature. To actuate the second actuation element 132b, heat/energy can be applied to the second target 138b. The heat applied to the second target 138b spreads through the second actuation element 132b, which can heat at least the portion of the second actuation element 132b above its transition temperature. In the illustrated embodiment, the targets 138 are positioned generally centrally along a length of each individual actuation element 132a-b. In other embodiments, however, the targets 138a-b can be positioned at an end region of each individual actuation element 132a-b. In some embodiments, the targets 138a-b are composed of a same material (e.g., nitinol) as the actuation elements 132a-b. Without being bound by theory, the increased surface area of the targets 138a-b relative to the actuation elements 132a-b is expected in increase the ease and consistency by which the actuators 130 can be actuated using an energy source (e.g., a laser) positioned external to the body.

In some embodiments the system 100 and/or the flow control assembly 120 can be configured to operate in reverse. For example, in at least some embodiments fluid can enter the system 100 via the one or more fluid outlets 106 (FIG. TA), the actuator 130 can control the flow of fluid into the chamber 121, and fluid can drain from the flow control assembly 120 via the fluid inlet 124. Additional details regarding the operation of shape memory actuators suitable for use with the present technology, as well as adjustable glaucoma shunts, are described in U.S. Pat. Nos. 11,058,581, 11,166,849, and 11,291,585, U.S. patent application Ser. No. 17/774,310, and International Patent Application Nos. PCT/US22/13336, PCT/US20/55144, PCT/US20/55141, PCT/US21/14774, PCT/US21/18601, PCT/US21/23238, PCT/US21/27742, and PCT/US21/49140, the disclosures of which are incorporated by reference herein in their entireties and for all purposes.

FIG. 1D is an exploded perspective view of the flow control assembly 120 of FIG. 1B. As illustrated, in some embodiments, the flow control assembly 120 can include one or more individual plates or layers 122. In the illustrated embodiment, for example, the flow control assembly 120 includes four plates 122a-d (e.g., a first plate 122a, a second plate 122b, a third plate 122c, and a fourth plate 122d). In other embodiments, however, the flow control assembly 120 can include more or fewer plates 122. Each of the plates 122a-d can be coupled to, at least partially aligned with, and/or otherwise positioned relative to (e.g., above, below, etc.) one or more of the other plates 122a-d. In the illustrated embodiment for example, the third plate 122c is positioned above and at least partially aligned with the fourth plate 122d, the second plate 122b is positioned above and at least partially aligned with the third plate 122c, and the first plate 122a is positioned above and at least partially aligned with the second plate 122b. In other embodiments, each of the plates 122a-d can have any other suitable alignment and/or position relative to each other. Each of the plates 122a-d can be coupled to one or more adjacent plates. In the illustrated embodiment, for example, a lower surface of the first plate 122a can be coupled to an upper surface of the second plate 122b, a lower surface of the second plate 122b can be coupled to an upper surface of the third plate 122c, and a lower surface of the third plate 122c can be coupled to the fourth plate 122d. The coupling between each adjacent plate can form a substantial fluid seal between the adjacent plates, such that fluid can enter (e.g., only enter) the plate assembly 120 via the fluid inlet 124 and/or the channel outlets 137a-b.

One or more of the plates 122a-d can include or at least partially define various features of the flow control assembly 120. In the illustrated embodiment, for example, the first plate 122a includes the fluid inlet 124 and an upper surface to the chamber 121; the second plate 122b includes the fluid inlet conduit 125, a volume of the chamber 121, and the actuator 130; and the third plate 122c includes the channel inlets 135a-b, the channels 136a-b, and the channel outlets 137a-b. In other embodiments, the individual plates or layers can include or define the various features of the flow control assembly 120 in other arrangements. In these and other embodiments, one or more of the features of the flow control assembly 120 can be distributed between a plurality of different (e.g., adjacent) plates 122. For example, in the illustrated embodiment, the fluid inlet conduit 125 is formed in an upper surface of the second plate 122b such that, when the second plate 122b is coupled to the first plate 122a, at least a portion of a lower surface of the first plate 122a forms an upper surface of the fluid inlet conduit 125.

Although the flow control assembly 120 in FIGS. 1A-1D is illustrated as having a single fluid inlet 124, chamber 121, and actuator 130, in other embodiments the flow control assembly 120 can include more fluid inlets, chambers, and/or actuators. For example, the flow control assembly 120 can include at least two, three, four, or any other suitable number of fluid inlets, chambers, and/or actuators. Although in FIGS. 1A-1C the flow control assembly 120 is illustrated as having two channels 136a-b, in other embodiments the flow control assembly 120 can include more channels. For example, the flow control assembly 120 can include at least three, four, five, or any other suitable number of channels 136. In these and other embodiments, the actuator 130 can be transitionable between at least as many positions as the number of channels 136, such that the actuator 130 can selectively interfere with fluid flow through each of the channels 136 individually, as described previously. In some embodiments, each actuator 130 may control the flow of fluid through two channels 136, such that there are twice as many channels 136 as actuators 130.

FIGS. 2A and 2B are circuit diagrams schematically illustrating the flow paths through the system 100. Specifically, FIG. 2A illustrates the system 100 in a first configuration in which the gating element (not shown) of the actuator 130 is in the first position (permitting fluid flow through the first channel 136a and blocking fluid flow through the second channel 136b), and FIG. 2B illustrates the system 100 in a second configuration in which the gating element (not shown) of the actuator 130 is in the second position (permitting fluid flow through the second channel 136b and blocking fluid flow through the first channel 136a). In FIGS. 2A and 2B, selected portions of the system 100 (e.g., the fluid inlet 124, the fluid inlet conduit 125, the actuator 130, the channels 136a-b, the main fluid conduit 110) are shown schematically using dashed-line boxes for the purpose of clarity.

Referring first to FIG. 2A, when the system 100 is in the first configuration, fluid can enter the system 100 via the fluid inlet 124 and travel through the fluid inlet conduit 125 toward the actuator 130. With the actuator 130 in the first position, the fluid flows through the first channel 136a toward the main fluid conduit 110 and drains from the system 100 via the outlet 106. Referring next to FIG. 2B, the operation of the system 100 can be generally similar to the operation described with reference to FIG. 2A. However, in FIG. 2B the actuator 130 has been transitioned to the second position such that the fluid within the system 100 flows through the second channel 136b rather than the first channel 136a.

Referring to FIGS. 2A and 2B together, each of the channels 136a-b can have a respective fluid resistance, as described previously. In the illustrated embodiment, for example, the first channel 136a has a first fluid resistance R₁ and the second channel 136b has a second fluid resistance R₂. The first fluid resistance R₁ is generally different (e.g., greater than or less than) the second fluid resistance R₂. Accordingly, in at least some embodiments, transitioning the actuator 130 from the first position (FIG. 2A) in which fluid primary flows through the first channel 136a to the second position (FIG. 2B) in which fluid primarily flows through the second channel 136b can change the overall fluid resistance of the system 100. In some embodiments, one or more other portions of the system 100 can have respective fluid resistances that affect the overall fluid resistance to flow through the system 100. For example, the fluid inlet conduit 125 has a third fluid resistance R₃ and the main fluid conduit 110 has a fourth fluid resistance R₄. The third fluid resistance R₃ and/or the fourth fluid resistance R₄ can each be less than, equal to, or greater than the first fluid resistance R₁ and/or the second fluid resistance R₂. In at least some embodiments, the third fluid resistance R₃ and/or the fourth fluid resistance R₄ can be insignificant or negligible, such that the respective fluid resistances R₁₋₂ of the channels 136a-b provide all or substantially all of the overall fluid resistance of the system 100. Accordingly, under a given pressure, the flow rate through the system 100 can vary based on the position of the actuator 130.

### Examples

Several aspects of the present technology are set forth in the following examples:

1. An adjustable shunting system for treating a patient, the adjustable shunting system comprising:
a first channel having a first channel inlet and a first fluid resistance;
a second channel having a second channel inlet and a second fluid resistance different than the first fluid resistance; and
a single actuator transitionable between at least (i) a first position in which a portion of the single actuator is at least partially aligned with the first channel inlet and (ii) a second position in which the portion of the single actuator is at least partially aligned with the second channel inlet.

2. The adjustable shunting system of example 1 wherein, in the first position, the portion of the single actuator at least partially blocks flow through the first channel inlet and permits flow through the second channel inlet.

3. The adjustable shunting system of example 1 or example 2 wherein, in the second position, the portion of the single actuator at least partially blocks flow through the second channel inlet and permits flow through the first channel inlet.

4. The adjustable shunting system of any of examples 1-3 wherein the first fluid resistance is less than the second fluid resistance.

5. The adjustable shunting system of any of examples 1-3 wherein the first fluid resistance is greater than the second fluid resistance.

6. The adjustable shunting system of any of examples 1-5 wherein the single actuator is transitionable to a third position in which the portion of the single actuator is aligned to have an offset from the first channel inlet or the second channel inlet.

7. The adjustable shunting system of example 6 wherein, in the third position, the portion of the single actuator is located between the first channel inlet and the second channel inlet.

8. The adjustable shunting system of example 6 wherein, in the third position, the portion of the single actuator allows flow through the first channel inlet and the second channel inlet.

9. The adjustable shunting system of any of examples 1-8, further comprising a third channel having a third fluid inlet and a third fluid resistance, wherein the single actuator is further transitionable to a third position in which the portion of the single actuator is at least partially aligned with the third fluid inlet.

10. The adjustable shunting system of example 9 wherein the third fluid resistance is different than the first fluid resistance or the second fluid resistance.

11. The adjustable shunting system of example 9 or example 10 wherein, in the third position, the portion of the actuator at least partially blocks flow through the third channel inlet and permits flow through at least one of the first channel inlet and the second channel inlet.

12. The adjustable shunting system of any of examples 1-11 wherein the portion of the actuator includes a gating element of the single actuator, and wherein:
the gating element at least partially blocks flow through the first channel inlet and permits flow through the second channel inlet when the single actuator is in the first position; and
the gating element at least partially blocks flow through the second channel inlet and permits flow through the first channel inlet when the single actuator is in the second position.

13. The adjustable shunting system of example 12 wherein the gating element includes a spherical portion.

14. The adjustable shunting system of example 13 wherein the spherical portion is configured to sealingly engage the first channel inlet and/or the second channel inlet.

15. The adjustable shunting system of example 12 wherein the gating element includes silicone.

16. The adjustable shunting system of any of examples 1-15, further comprising a flow control assembly, the flow control assembly defining the first channel, the second channel, and a chamber configured to receive the single actuator.

17. The adjustable shunting system of example 16, wherein:
the first channel is fluidly coupled to the chamber by the first channel inlet; and
the second channel is fluidly coupled to the chamber by the second channel inlet.

18. The adjustable shunting system of example 16 or example 17 wherein the chamber is positioned within an interior of the flow control assembly, the flow control assembly further comprising a fluid inlet fluidly coupled to the chamber and configured to permit fluid from an environment external to the flow control assembly to enter the chamber.

19. The adjustable shunting system of example 18, further comprising a fluid inlet conduit fluidly coupling the fluid inlet and the chamber.

20. The adjustable shunting system of example 19 wherein the fluid inlet conduit has a third fluid resistance less than the first fluid resistance and/or the second fluid resistance.

21. The adjustable shunting system of example 20 wherein the fluid inlet conduit has a third fluid resistance greater than or equal to the first fluid resistance or the second fluid resistance.

22. The adjustable shunting system of any of examples 16-21 wherein the flow control assembly comprises at least one plate, the at least one plate including a first plate defining the first channel and the second channel.

23. The adjustable shunting system of example 22 wherein the flow control assembly further comprises a second plate including the single actuator and at least partially defining the chamber.

24. The adjustable shunting system of example 23 wherein the flow control assembly further comprises a third plate including a fluid inlet fluidly coupled to the chamber and configured to permit fluid from an environment external to the flow control assembly to enter the chamber.

25. The adjustable shunting system of example 24 wherein the first plate is positioned on a first side of the second plate and the third plate is positioned on a second side of the second plate, the second side opposite the first side.

26. The adjustable shunting system of any of examples 1-25 wherein the single actuator includes:
a first actuation element operable to transition the single actuator from the first position toward the second position; and
a second actuation element operable to transition the single actuator from the second position toward the first position.

27. The adjustable shunting system of example 26 wherein the first actuation element and the second actuation element are composed of a shape memory material.

28. A method for selectively controlling fluid flow through a shunting system implanted within a patient, the method comprising:
adjusting a fluid resistance through the system by applying energy to an actuation element of an actuator of the shunting system,
wherein applying energy to the actuation element causes the actuator to move between (i) a first position in which the actuator at least partially blocks flow through a first channel of the system and permits flow through a second channel of the system, and (ii) a second position in which the actuator at least partially blocks flow through the second channel and permits flow through the first channel, and
wherein the first channel has a first fluid resistance and the second channel has a second fluid resistance different than the first fluid resistance.

29. The method of example 28 wherein adjusting the fluid resistance includes increasing the fluid resistance from the second fluid resistance to the first fluid resistance.

30. The method of example 28 wherein adjusting the fluid resistance includes decreasing the fluid resistance from the second fluid resistance to the first fluid resistance.

31. The method of any of examples 28-30 wherein the actuation element is a first actuation element, the method further comprising adjusting the fluid resistance by applying energy to a second actuation element of the actuator, wherein applying energy to the second actuation element causes the actuator to move from the second position to the first position.

32. The method of any of examples 28-31 wherein causing the actuator to move from the first position to the second position includes causing a gating element of the actuator to move from (i) a first orientation in which the gating element at least partially blocks flow through the first channel and permits flow through the second channel to (ii) a second orientation in which the gating element at least partially blocks flow through the second channel and permits flow through the first channel.

33. The method of example 32 wherein causing the gating element to move from the first orientation to the second orientation includes causing at least a portion of the gating element to be at least partially aligned with a fluid inlet of the second channel.

34. The method of any of examples 28-33 wherein applying energy includes applying laser energy from an energy source external to the patient.

35. The method of any of examples 28-34 wherein applying energy to the actuation element includes applying energy to a target region of the actuation element.

36. An adjustable shunting system for treating a patient, the adjustable shunting system comprising:
a fluid inlet;
a first channel fluidly coupled to the fluid inlet, the first channel having a first channel inlet and a first fluid resistance;
a second channel fluidly coupled to the fluid inlet, the second channel having a second channel inlet and a second fluid resistance different than the first fluid resistance; and
a single actuator transitionable between at least (i) a first position in which a portion of the single actuator is at least partially aligned with the first channel inlet and (ii) a second position in which the portion of the single actuator is at least partially aligned with the second channel inlet, wherein the single actuator is positioned between the fluid inlet and at least one of the first channel and the second channel.

37. The adjustable shunting system of example 36 wherein the single actuator is positioned above at least one of the first channel and the second channel.

38. The adjustable shunting system of example 36 wherein the single actuator is positioned between the fluid inlet and at least one of the first channel inlet and/or the second channel inlet.

39. The adjustable shunting system of any of examples 36-38, further comprising a chamber fluidly coupled to the fluid inlet and configured to receive fluid therefrom, wherein the single actuator is positioned within the chamber, and wherein, in at least one of the first position or the second position, the portion of the single actuator is configured to at least partially prevent the fluid within the chamber from flowing through at least one of the first channel inlet or the second channel inlet.

40. A method for selectively controlling fluid flow through a shunting system implanted within a patient, the method comprising:
adjusting a fluid resistance through the system by applying energy to an actuation element of an actuator of the shunting system,
wherein applying energy to the actuation element causes the actuator to move between (i) a first position in which the actuator is out of alignment with and does not impede fluid flow through both a first channel of the system and a second channel of the system, and (ii) a second position in which the actuator at least partially blocks fluid flow through the first channel or the second channel, and
wherein the first channel has a first fluid resistance and the second channel has a second fluid resistance different than the first fluid resistance.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An adjustable shunting system for treating a patient, the adjustable shunting system comprising:
a first channel having a first channel inlet and a first fluid resistance;
a second channel having a second channel inlet and a second fluid resistance different than the first fluid resistance; and
a single actuator transitionable between at least (i) a first position in which a portion of the single actuator is at least partially aligned with the first channel inlet and (ii) a second position in which the portion of the single actuator is at least partially aligned with the second channel inlet.

2. The adjustable shunting system of claim 1 wherein, in the first position, the portion of the single actuator at least partially blocks flow through the first channel inlet and permits flow through the second channel inlet.

3. The adjustable shunting system of claim 1 wherein, in the second position, the portion of the single actuator at least partially blocks flow through the second channel inlet and permits flow through the first channel inlet.

4. The adjustable shunting system of claim 1 wherein the first fluid resistance is less than the second fluid resistance.

5. The adjustable shunting system of claim 1 wherein the first fluid resistance is greater than the second fluid resistance.

6. The adjustable shunting system of claim 1 wherein the single actuator is transitionable to a third position in which the portion of the single actuator is aligned to have an offset from the first channel inlet or the second channel inlet.

7. The adjustable shunting system of claim 6 wherein, in the third position, the portion of the single actuator is located between the first channel inlet and the second channel inlet.

8. The adjustable shunting system of claim 6 wherein, in the third position, the portion of the single actuator allows flow through the first channel inlet and the second channel inlet.

9. The adjustable shunting system of claim 1, further comprising a third channel having a third fluid inlet and a third fluid resistance, wherein the single actuator is further transitionable to a third position in which the portion of the single actuator is at least partially aligned with the third fluid inlet.

10. The adjustable shunting system of claim 9 wherein the third fluid resistance is different than the first fluid resistance or the second fluid resistance.

11. The adjustable shunting system of claim 9 wherein, in the third position, the portion of the actuator at least partially blocks flow through the third channel inlet and permits flow through at least one of the first channel inlet and the second channel inlet.

12. The adjustable shunting system of claim 1 wherein the portion of the actuator includes a gating element of the single actuator, and wherein:
the gating element at least partially blocks flow through the first channel inlet and permits flow through the second channel inlet when the single actuator is in the first position; and
the gating element at least partially blocks flow through the second channel inlet and permits flow through the first channel inlet when the single actuator is in the second position.

13. The adjustable shunting system of claim 12 wherein the gating element includes a spherical portion.

14. The adjustable shunting system of claim 13 wherein the spherical portion is configured to sealingly engage the first channel inlet and/or the second channel inlet.

15. The adjustable shunting system of claim 12 wherein the gating element includes silicone.

16. The adjustable shunting system of claim 1, further comprising a flow control assembly, the flow control assembly defining the first channel, the second channel, and a chamber configured to receive the single actuator.

17. The adjustable shunting system of claim 16, wherein:
the first channel is fluidly coupled to the chamber by the first channel inlet; and
the second channel is fluidly coupled to the chamber by the second channel inlet.

18. The adjustable shunting system of claim 16 wherein the chamber is positioned within an interior of the flow control assembly, the flow control assembly further comprising a fluid inlet fluidly coupled to the chamber and configured to permit fluid from an environment external to the flow control assembly to enter the chamber.

19. The adjustable shunting system of claim 18, further comprising a fluid inlet conduit fluidly coupling the fluid inlet and the chamber.

20. The adjustable shunting system of claim 19 wherein the fluid inlet conduit has a third fluid resistance less than the first fluid resistance and/or the second fluid resistance.

21. The adjustable shunting system of claim 20 wherein the fluid inlet conduit has a third fluid resistance greater than or equal to the first fluid resistance or the second fluid resistance.

22. The adjustable shunting system of claim 16 wherein the flow control assembly comprises at least one plate, the at least one plate including a first plate defining the first channel and the second channel.

23. The adjustable shunting system of claim 22 wherein the flow control assembly further comprises a second plate including the single actuator and at least partially defining the chamber.

24. The adjustable shunting system of claim 23 wherein the flow control assembly further comprises a third plate including a fluid inlet fluidly coupled to the chamber and configured to permit fluid from an environment external to the flow control assembly to enter the chamber.

25. The adjustable shunting system of claim 24 wherein the first plate is positioned on a first side of the second plate and the third plate is positioned on a second side of the second plate, the second side opposite the first side.

26. The adjustable shunting system of claim 1 wherein the single actuator includes:
a first actuation element operable to transition the single actuator from the first position toward the second position; and
a second actuation element operable to transition the single actuator from the second position toward the first position.

27. The adjustable shunting system of claim 26 wherein the first actuation element and the second actuation element are composed of a shape memory material.

28. A method for selectively controlling fluid flow through a shunting system implanted within a patient, the method comprising:
adjusting a fluid resistance through the system by applying energy to an actuation element of an actuator of the shunting system,
wherein applying energy to the actuation element causes the actuator to move between (i) a first position in which the actuator at least partially blocks flow through a first channel of the system and permits flow through a second channel of the system, and (ii) a second position in which the actuator at least partially blocks flow through the second channel and permits flow through the first channel, and
wherein the first channel has a first fluid resistance and the second channel has a second fluid resistance different than the first fluid resistance.

29. The method of claim 28 wherein adjusting the fluid resistance includes increasing the fluid resistance from the second fluid resistance to the first fluid resistance.

30. The method of claim 28 wherein adjusting the fluid resistance includes decreasing the fluid resistance from the second fluid resistance to the first fluid resistance.

31. The method of claim 28 wherein the actuation element is a first actuation element, the method further comprising adjusting the fluid resistance by applying energy to a second actuation element of the actuator, wherein applying energy to the second actuation element causes the actuator to move from the second position to the first position.

32. The method of claim 28 wherein causing the actuator to move from the first position to the second position includes causing a gating element of the actuator to move from (i) a first orientation in which the gating element at least partially blocks flow through the first channel and permits flow through the second channel to (ii) a second orientation in which the gating element at least partially blocks flow through the second channel and permits flow through the first channel.

33. The method of claim 32 wherein causing the gating element to move from the first orientation to the second orientation includes causing at least a portion of the gating element to be at least partially aligned with a fluid inlet of the second channel.

34. The method of claim 28 wherein applying energy includes applying laser energy from an energy source external to the patient.

35. The method of claim 28 wherein applying energy to the actuation element includes applying energy to a target region of the actuation element.

36. An adjustable shunting system for treating a patient, the adjustable shunting system comprising:
a fluid inlet;
a first channel fluidly coupled to the fluid inlet, the first channel having a first channel inlet and a first fluid resistance;
a second channel fluidly coupled to the fluid inlet, the second channel having a second channel inlet and a second fluid resistance different than the first fluid resistance; and
a single actuator transitionable between at least (i) a first position in which a portion of the single actuator is at least partially aligned with the first channel inlet and (ii) a second position in which the portion of the single actuator is at least partially aligned with the second channel inlet, wherein the single actuator is positioned between the fluid inlet and at least one of the first channel and the second channel.

37. The adjustable shunting system of claim 36 wherein the single actuator is positioned above at least one of the first channel and the second channel.

38. The adjustable shunting system of claim 36 wherein the single actuator is positioned between the fluid inlet and at least one of the first channel inlet and/or the second channel inlet.

39. The adjustable shunting system of claim 36, further comprising a chamber fluidly coupled to the fluid inlet and configured to receive fluid therefrom, wherein the single actuator is positioned within the chamber, and wherein, in at least one of the first position or the second position, the portion of the single actuator is configured to at least partially prevent the fluid within the chamber from flowing through at least one of the first channel inlet or the second channel inlet.

40. A method for selectively controlling fluid flow through a shunting system implanted within a patient, the method comprising:
adjusting a fluid resistance through the system by applying energy to an actuation element of an actuator of the shunting system,
wherein applying energy to the actuation element causes the actuator to move between (i) a first position in which the actuator is out of alignment with and does not impede fluid flow through both a first channel of the system and a second channel of the system, and (ii) a second position in which the actuator at least partially blocks fluid flow through the first channel or the second channel, and
wherein the first channel has a first fluid resistance and the second channel has a second fluid resistance different than the first fluid resistance.
